(19) United States
(12) Patent Application Publication
(10) Pub. No.: US 2005/0124960 A1
(43) Pub. Date: Jun. 9, 2005

(54) Combination pouch/backing sheet for personal care absorbent articles with mechanical attachment system

(75) Inventor: Marcille F. Ruman, Oshkosh, WI (US)

Correspondence Address: DORITY & MANNING, P.A., POST OFFICE BOX 1449, GREENVILLE, SC, 29602-1449, US

(73) Assignee: KIMBERLY-CLARK WORLDWIDE, INC.

(21) Appl. No.: 10/731,685

(22) Filed: Dec. 9, 2003

(51) Int. Cl. A61F 13/15; A61F 13/20

(52) U.S. Cl. 604/385.19

(57) ABSTRACT

An absorbent article, such as a feminine care product, includes a liquid permeable top cover, a generally liquid impermeable baffle, and an absorbent structure disposed between the top cover and outer cover. The garment facing side of the baffle includes hook material as a primary attachment mechanism between the baffle and the undergarment. A hook compatible backing sheet is releasably attached to the baffle. The article and attached backing sheet are folded into a configuration such that lateral sides of the backing sheet are brought together and the backing sheet defines a pouch for the article.

### FIELD OF THE INVENTION

The present invention relates generally to the field of disposable absorbent articles, and more particularly to a method for packaging feminine care articles.

### BACKGROUND

Disposable absorbent articles intended to be worn in the crotch portion of an undergarment are well known. The basic form of such absorbent articles typically includes a bodyside liquid-permeable cover, a garment-side liquid-impermeable baffle, and an absorbent core positioned between the cover and the baffle. Such absorbent articles are now in wide use as sanitary napkins, panty shields, panty liners, and adult incontinence pads. While this invention is directed to all such products, for purposes of simplification, the invention will be described with reference to a sanitary napkin.

Present day commercial products have generally performed well, remaining in place during use and providing the user with ease of placement and removal. Such products typically use an adhesive strip applied to the garment facing side of the baffle for attachment of the article to the wearer's undergarment. The adhesive strip is covered with a peel strip that is removed by the wearer prior to placing the article in the crotch region of the undergarment. These products are typically folded, for example in a tri-fold configuration, and packaged in individual disposable pouches or the like. With certain commercial products, the peel strip also functions as the pouch. The strip is of a size and shape such that, upon folding the article, the peel strip forms an individual pouch with sides that are subsequently bonded and a flap that may also be adhered to the pouch. For aesthetic and functionality purposes, the typical pouch material is a poly film material. The combination pouch/peel strip configuration provides an efficient and relatively cost effective method for individually wrapping each article and eliminates the need for separate pouch material and release liner. Reference is made, for example, to U.S. Pat. No. 6,234,229 B1; U.S. Pat. No. 6,312,417 B1; U.S. Pat. No. 5,474,818; and U.S. Pat. No. 5,181,610.

Although convenient, the use of adhesives for attaching the articles to a wearer's undergarment presents certain inherent drawbacks. For example, the inner crotch surface of an undergarment, to which these products are typically adhered, is continually being distorted, twisted and stretched due to the dynamics of the wearer. As a result, conventional adhesive attachments can detach causing the undesirable consequence of the sanitary napkin moving out of place. Further, while the sanitary napkin frequently reattaches itself, due to the continuing adhesive nature of the pressure sensitive adhesive, reattachment often places the sanitary napkin in an undesirable position wherein the article does not function properly. Additionally, once the adhesives have been exposed and the article placed into the crotch region of the undergarment, it is generally not practical to remove and reposition the article. In an extreme case, the attachment of the adhesive also results in the adhesive folding over on itself and then becoming unavailable for reattachment.

Attention has thus been given in the art to the use of mechanical fastening systems, such as hook-and-loop systems, for the purpose of attaching the articles directly to the undergarment. For example, U.S. Pat. No. 5,300,058 describes a sanitary napkin that uses an oriented hook fastening material on the article baffle to secure the article in place. Similarly, U.S. Pat. No. 5,611,790 describes an extensible absorbent article that may use mechanical hook material for securing the article to a garment. U.S. Pat. No. 5,778,457 also describes an absorbent pad utilizing a hook material as the fastening mechanism. Reference is also made to the following U.S. patents for various mechanical fastening systems: U.S. Pat. Nos. 6,393,621; 6,077,255; 5,795,349; 5,779,692; 5,762,645; 5,676,652; and 5,584,829.

The industry is continually seeking ways to improve personal care absorbent articles and, in this light, articles that offer the benefits of an improved mechanical attachment mechanism that compliments a more beneficial and economic packaging process would be desirable. The present invention provides such an improved product.

### SUMMARY

Objects and advantages of the invention will be set forth in part in the following description, or may be obvious from the description, or may be learned through practice of the invention.

Although the present invention has particular usefulness in the field of feminine care articles such as panty shields or liners and sanitary napkins, it should be appreciated that any manner of personal care absorbent article may benefit from the invention, including incontinence articles, and the like. All such uses are within the scope and spirit of the invention. For ease of description only, the working environment of the invention is assumed to be feminine care sanitary napkins.

In accordance with the invention, a packaged absorbent article, such as a panty shield or sanitary napkin, is provided for placement in the crotch region of a wearer's undergarment. The absorbent article includes a generally liquid permeable top cover, such as any one or combination of nonwoven materials know in the art as suitable for absorbent article cover materials. A generally liquid impermeable baffle is provided having a garment facing side. The baffle may be, for example, a film material. An absorbent structure is disposed between the top cover and the baffle. Suitable absorbent materials are well known to those skilled in the art.

The garment facing side of the baffle includes hook material defined in a pattern such that the hook material provides a primary attachment mechanism between the article and the undergarment by way of the wearer simply pressing the garment facing side of the baffle against the undergarment. Conventional undergarment materials, such as natural or synthetic fiber woven or nonwoven materials, inherently function as a “loop” or “hook compatible material” when engaged by conventional hook or micro-hook materials and, thus, the baffle hook material releasably attaches to the undergarment. As described in greater detail below, the pattern and type of baffle hook material may vary widely within the scope and spirit of the invention.

A hook compatible backing sheet is releasably attached to the garment facing side of the baffle and may have dimensions so as to extend beyond the lateral sides and longitudinal ends of the absorbent article when attached to the baffle. For example, in one particular embodiment, the backing sheet generally defines a border around the baffle. The backing sheet may be formed wholly or in part by a nonwoven material so as to cover and engage with the hook material on the garment facing side of the baffle. The article and attached backing sheet are folded into a configuration such that the backing sheet defines a pouch-like structure for the absorbent article. The size and shape of the pouch may vary as a function of the size of the backing sheet, fold pattern, etc. In one particular embodiment, the lateral sides of the backing sheet are brought together in the folded configuration and may be sealed by conventional means. In a tri-fold configuration, one of the longitudinal ends of the backing sheet and attached article may be folded over towards a front surface of the pouch so as to define an opening flap. This flap may be sealed or otherwise attached to the front surface of the pouch.

It should thus be understood that the backing sheet serves various functions. By releasably attaching to the baffle hook material, the backing sheet protects the top cover from being engaged by the hook material when the article is folded or unfolded by the wearer prior to use. Engagement of the hook material with the cover is undesirable in that it may result in picking of the cover material and give an overall impression of lesser quality to the wearer. The backing sheet also protects the cover from being torn or otherwise damaged by the hook material. Tears or holes in the cover material could affect the article performance and are undesirable. For example, holes or tears in the cover could affect cover dryness, fluid intake and retention, or even lead to exposed absorbent. The backing sheet additionally provides a means for defining a convenient and discrete individual pouch for the article. For use, the wearer simply unfolds the pouch, peels the pouch material (backing sheet) from the article, and presses the article into place in the crotch region of the undergarment.

In a particular embodiment, the backing sheet may be formed from two or more different types of material. For example, the backing sheet may have a first side of a hook compatible material and an opposite side of a poly film material. The sheet may be a laminate of these materials. The hook compatible material side engages with the baffle hook material and the poly film side defines the outer surface of the pouch. The poly film may be desirable for functionality and aesthetic purposes. The film may give the pouch a higher degree of liquid impermeability, and provide a suitable material for disposal of soiled articles. The presence of a film material may, however, add to the noise generated when opening the pouch.

In an alternate embodiment, the entire article facing side of the backing sheet need not be formed from hook compatible material. For example, the backing sheet may be formed substantially of the poly film material with a relatively small strip (i.e., a center longitudinally disposed strip) of hook compatible material attached thereto just sufficient for covering the baffle hook material.

With still an alternate embodiment, the backing sheet may be formed of a nonwoven material having zones of different engagement strength. For example strips or zones of a nonwoven material may be altered (mechanically, thermally, ultrasonically, and so forth) so as to more readily attach to the hook material. Alternately, the backing sheet may be formed of a nonwoven material wherein the engagement properties of the material differ from one side to another.

The invention is also applicable to absorbent articles that incorporate laterally extending wings configured to fold around the crotch portion of a wearer's undergarment. The wings may be folded onto the top cover in the folded configuration of the article such that the garment facing sides of the wings are outwardly facing. The wings may have hook material provided in a pattern on the garment facing side for attachment to the undergarment or to each other. In this case, a protective material sheet may be attached to the wing hook material prior to folding the article and backing sheet so that the wing hook material does not engage with the top cover. In an alternate embodiment, the wings may incorporate adhesive patches or deposits for attachment to each other or the undergarment. A peel strip may be provided on the adhesive regions. The peel strip or protective material strip would also serve to hold the wings in place until the pad is applied to the undergarment, at which point the strip is removed and the wings are folded around the edges of the undergarment. In an alternate embodiment, the backing sheet may have a substantial longitudinal length as compared to the article (with wings) and, once the article is placed on the backing sheet, an end of the sheet may be folded over the article and cover the wings. The combination may then be folded into a pouch configuration, as discussed.

As mentioned, with the combination pouch/backing sheet configuration also provides a means to discretely dispose of soiled articles without the need for disposal wrappers, pouches, or the like, in additional to a peel strip. After unfolding the pouch and peeling the backing sheet from the article, the wearer can simply grasp the soiled article by the edges, press it against the backing sheet and fold the combination into a disposal pouch. A poly film sides backing sheet may be desirable in that it may reduce odor from the soiled product. Also, a sealing feature may be provided on the backing sheet, such as an adhesive patch, peel tape, hook-and-loop fastener, etc., to hold the pouch (with soiled product) closed. This sealing feature may be the same reusable device that holds the pouch (with a clean new product) closed, or may be a different device.

The pattern of hook material defined on the baffle may be any desired continuous or discontinuous placement pattern, for example stripes, dashes, swirls, dots or island-like regions, etc. The amount and pattern of the hook material should be sufficient to ensure that the baffle remains secured in the crotch region of the undergarment. In a desirable embodiment, the garment facing side of the baffle may present an overall uniform pattern of hook material generally over its entire surface area such that a uniform vapor permeability gradient is established. For example, the vapor permeability gradient of a transverse section of the article at one longitudinal end is generally the same for a comparable transverse section in the medial or opposite longitudinal end of the article. The garment will thus be retained in the undergarment more securely than if a single area or region of conventional adhesive were used, such as a longitudinal strip of adhesive along the center of the baffle as with many types of commercial articles. Yet, even with such improved attachment, the article remains breathable and is easily removable and variably positionable as required by the wearer.

There is a desirable range of engagement between the baffle hook material and backing sheet that may be empirically determined depending on the shape and type of the hooks, type of backing sheet material, shape of the absorbent article, and so forth.

The hook material may be deposited on the baffle by conventional methods, including by applying strips of hook material tape with adhesive, etc. In a particularly desirable embodiment, the hook material is extruded and laminated directly onto a base material used for the baffle, as described in greater detail below.

Aspects of the invention will described below in greater detail by reference to particular embodiments, examples of which are illustrated in the figures.

### BRIEF DESCRIPTION OF THE FIGURES

FIG. 1 is a perspective view of an absorbent article according to the invention with the backing sheet being placed onto the baffle.

FIG. 2 is a top plan view of the absorbent article according to FIG. 1 after placement of the backing sheet.

FIG. 3 is a perspective view of the absorbent article according to FIG. 2 being folded into a tri-fold configuration.

FIG. 4 is a perspective view of the pouch configuration of the backing sheet.

FIG. 5 is a perspective and partial cut-away view of an alternate embodiment of an absorbent article according to the invention particularly illustrating an alternate backing sheet material.

FIG. 6 is a perspective and partial cut-away view of another embodiment of an absorbent article according to the invention illustrating a different embodiment of the backing sheet material.

FIG. 7 is a top plan view of an absorbent article combination according to the invention illustrating the article wings folded onto the top cover prior to folding the backing sheet.

FIG. 8 is a top plan view of an alternate embodiment of a winged folded article.

FIG. 9 is a top plan view of still another embodiment of a winged folded article place on a backing sheet.

FIGS. 10A through 10C are top plan views and a perspective view of an alternate absorbent article combination according to the invention.

### DETAILED DESCRIPTION

The invention will now be described in detail with reference to particular embodiments thereof. The embodiments are provided by way of explanation of the invention, and are not meant as a limitation of the invention. For example, features described or illustrated as part of one embodiment may be used with another embodiment to yield still a further embodiment. It is intended that the present invention include these and other modifications and variations as come within the scope and spirit of the invention.

Referring to the Figures, in which like numerals indicate like parts throughout the several views, embodiments of a disposable absorbent article 10 are depicted. For purposes of illustration only, the disposable absorbent article 10 is exemplified as a sanitary napkin. Typically, a sanitary napkin is worn by a female to absorb body fluids, such as menses, blood, urine and other body excrements discharged during a menstrual period. Although the invention has particular usefulness for feminine care articles and, it should be appreciated that the invention is in no way limited to sanitary napkins in particular, or to feminine care articles in general. One skilled in the art will readily understand the adaptability of the invention to other personal care and health care articles, such as, for example, panty liners, adult incontinence garments and the like that attach to a wearer's undergarment.

The term “disposable”, as used herein, means that the absorbent article is discarded after a single use and is not intended to be laundered for subsequent reuse.

Referring to the figures in general, a package configuration 18 according to the invention is illustrated as an absorbent article 10 with a backing sheet 24 releasably attached to a baffle 14 of the article 10. The backing sheet 24 is foldable into a pouch 38 after attachment to the article 10, as described in greater detail below. The article 10 may be of conventional formation and includes a generally liquid permeable top cover 12, a generally liquid impermeable and vapor permeable outer cover or baffle 14, and an absorbent structure 20 disposed between the top cover 12 and baffle 14. The top cover 12 and baffle 14 are sealed together at their peripheral edges utilizing known techniques, such as, for example, gluing, crimping, hot-sealing or the like, the sealed edges defining an overall sealed peripheral edge. The article 10 may take on various shapes, but will generally have opposite longitudinal ends A, B and lateral sides C, D. In the illustrated embodiments, the article 10 has a well-known hourglass shape. Various other geometries of absorbent articles, including feminine care articles, are well known to those skilled in the art, and all such embodiments are within the scope and spirit of the invention.

The absorbent article 10 is desirably provided with sufficient capacity to absorb and retain the intended amount and type of bodily exudate(s). The absorbent capacity is provided by the fluid retentive absorbent structure or web 20. The absorbent structure 20 can be any structure or combination of components which are generally compressible, conformable, non-irritating to a wearer's skin, and capable of absorbing and retaining liquids and certain body wastes. For example, the structure 20 may include an absorbent web material of cellulosic fibers (e.g., wood pulp fibers), other natural fibers, synthetic fibers, woven or nonwoven sheets, scrim netting or other stabilizing structures, superabsorbent material, binder materials, surfactants, selected hydrophobic and hydrophilic materials, pigments, lotions, odor control agents or the like, as well as combinations thereof. In a particular embodiment, the absorbent web material is a matrix of cellulosic fluff, and may also include superabsorbent hydrogel-forming particles. The cellulosic fluff may comprise a blend of wood pulp fluff. One preferred type of fluff is identified with the trade designation NB 416, available from Weyerhaeuser Corp., and is a bleached, highly absorbent wood pulp containing primarily soft wood fibers. The absorbent materials may be formed into a web structure by employing various conventional methods and techniques. For example, the absorbent web may be formed with a dry-forming technique, an air forming technique, a wet-forming technique, a foam-forming technique, or the like, as well as combinations thereof. The absorbent material may be a foam or foam-like material. Methods and apparatus for carrying out such techniques are well known in the art.

The absorbent structure 20 can contain superabsorbent materials which are effective in retaining body fluids. As a general rule, the superabsorbent material is present in the absorbent web in an amount of from about 0 to about 90 weight percent based on total weight of the web. Superabsorbents have the ability to absorb a large amount of fluid in relation to their own weight. Typical Superabsorbents used in absorbent articles, such as sanitary napkins, can absorb anywhere from 5 to 60 times their weight in body fluids. Superabsorbent materials are well known in the art and can be selected from natural, synthetic, and modified natural polymers and materials.

The absorbent web material may also be a coform material. The term “coform material” generally refers to composite materials comprising a mixture or stabilized matrix of thermoplastic fibers and a second non-thermoplastic material. Some examples of such coform materials are disclosed in U.S. Pat. No. 4,100,324 to Anderson, et al.; U.S. Pat. No. 5,284,703 to Everhart. et al.; and U.S. Pat. No. 5,350,624 to Georger, et al.; which are incorporated herein in their entirety by reference thereto for all purposes.

The absorbent web material utilized in the absorbent structure 20 is also selected so that the individual absorbent structure possesses a particular individual total absorbency depending on the intended article of use. For example, for infant care products, the total absorbency can be within the range of about 200-900 grams of 0.9 wt % saline, and can typically be about 500 g of saline. For adult care products, the total absorbency can be within the range of about 400-2000 grams of saline, and can typically be about 1300 g of saline. For feminine care products, the total absorbency can be within the range of about 7-50 grams of menstrual fluid, and can typically be within the range of about 30-40 g of menstrual fluid.

The absorbent structure 20 may be a multi-component and may include, for example, an intake layer or transfer delay layer in combination with the underlying absorbent web. Such configurations are well known to those skilled in the art.

The fluid permeable top cover 12 has an outwardly facing surface that may contact the body of the wearer and receive bodily exudate(s). The top cover 12 desirably is made of a material which is flexible and non-irritating to the wearer. As used herein, the term “flexible” is intended to refer to materials which are compliant and readily conform to the bodily surface(s) with which such materials are in contact, or materials which respond by easily deforming in the presence of external forces.

The top cover 12 is provided for comfort and conformability and functions to direct bodily exudate(s) away from the body, through the top cover 12 and toward the absorbent structure 20. The top cover 12 should retain little or no liquid in its structure so that the cover provides a relatively comfortable and non-irritating surface next to the tissues within the vestibule of a female wearer. The top cover 12 can be constructed of any woven or nonwoven material which is easily penetrated by bodily fluids which contact the surface of the cover. Examples of suitable cover materials include rayon, bonded carded webs of polyester, polypropylene, polyethylene, nylon, or other heat-bondable fibers, polyolefins, such as copolymers of polypropylene and polyethylene, linear low-density polyethylene, and aliphatic esters such as polylactic acid. Finely perforated film webs and net material can also be used. The cover may be apertured to increase its fluid intake capacity. A specific example of a suitable cover material is a bonded carded web made of polypropylene and polyethylene such as that used as cover stock for KOTEX® pantiliners and obtainable from Sandler Corporation, Germany. Other examples of suitable materials are composite materials of polymer and nonwoven fabric materials. The composite materials are typically in the form of integral sheets generally formed by the extrusion of a polymer onto a web of spunbonded material. The fluid permeable cover 12 can also contain a plurality of apertures formed therein which are intended to increase the rate at which bodily fluid(s) can penetrate through the cover and into the absorbent structure 20.

The top cover 12 may also be embossed with any desired embossing pattern to define embossed channels. Embossing techniques are well known to those skilled in the art. An embossing pattern not only creates an aesthetically pleasing surface, the channels facilitate intake of menses fluid. Menses will tend to flow along the densified edges of the channels rather than pool on contact points of the top cover 12.

The top cover 12 can be maintained in secured relation with the absorbent structure 20 by bonding all or a portion of the adjacent surfaces to one another. A variety of bonding methods known to one of skill in the art can be utilized to achieve any such secured relationship. Examples of such methods include, but are not limited to, the application of adhesives in a variety of patterns between the two adjoining surfaces, entangling at least portions of the adjacent surface of the absorbent with portions of the adjacent surface of the cover, or fusing at least portions of the adjacent surface of the cover to portions of the adjacent surface of the absorbent.

The baffle 14 has an outer garment facing side 16 and may be any one of a number of suitable liquid impermeable materials known in the art for use as outer covers or baffles in absorbent articles. Preferably, the baffle 14 will permit the passage of air and moisture vapor out of the article 10 while blocking the passage of body fluids. A suitable material is a micro-embossed polymeric film, such as polyethylene or polypropylene, having a thickness of about 0.025 to 0.13 millimeters. Bicomponent films can also be used, as well as woven and nonwoven fabrics which have been treated to render them liquid impermeable. A specific example of a baffle material is a polyethylene film such as that used in KOTEX® pantiliners and obtainable from Pliant Corporation, Schaumburg, Ill., USA. The cover can be maintained in secured relation with the absorbent structure 20 by bonding all or a portion of the adjacent surfaces to one another. A variety of bonding methods known to one of skill in the art can be utilized to achieve any such secured relation. Examples of such methods include, but are not limited to, ultrasonic bonding, thermal bonding, or the application of adhesive materials in a variety of patterns between the two adjoining surfaces.

As illustrated generally in FIGS. 7 through 9, the article 10 may include laterally extending wings 48 that aid in securing the article 10 to the wearer's undergarment. Such wings are well know in the art and generally function by folding around the edges of the undergarment crotch region and attaching to the wearer's undergarment or to each other. A detailed explanation of such wing structure is not necessary for purposes of explanation of the present invention.

A hook material 22 is provided in a pattern on the garment facing side 16 of the baffle 14. The particular design, shape, etc., of the pattern are not limiting features. The pattern should provide sufficient coverage of hook material 22 to ensure reliable attachment of the article 10 to the wearer's undergarment. Undergarments are typically made from various woven or non-woven materials that present an attachment surface for conventional hook materials. Thus, the hook material 22 on the garment facing side 16 of the baffle 14 may attach directly to the inner or body facing side of the wearer's undergarment, and can provide the primary attachment mechanism between the article 10 and the garment.

The amount of the hook material 22 relative to the entire surface area of the body facing side 16 may vary between about 5% to about 75%, with desirable coverage being about 10% to about 50% of the surface area. In a particular embodiment, the coverage is about 25%. Desirably, the amount of coverage of the hook material 22 should be such that at least about 50% of the baffle 14 is not covered by hook material and remains vapor permeable.

As mentioned, the pattern of the hook material 22 may vary, but is desirably defined by deposits of hook material 22 interspaced with “bare” regions of the baffle material 14. The deposits of hook material 22 may be generally longitudinally oriented as continuous or discontinuous placements between the longitudinal ends A and B such that the vapor permeability gradient of the baffle 14 is generally uniform over the surface area of the baffle 14. For example, the permeability of a random transverse section of the article is generally uniform over the length of the article. Laterally oriented deposits of the hook material 22 extending generally between the lateral sides C and D are also within the scope and spirit of the invention. In a particular embodiment illustrated in the figures, the hook material 22 is defined in a pattern of continuous longitudinal stripes interspaced with bands of bare baffle material. The surface area of the hook material 22 may be, for example, about one-half of that of the bare regions of the baffle material 14.

In an alternate embodiment, the pattern of hook material 22 may be defined in different complementary patterns. For example, the hook material 22 may be defined in a pattern of discontinuous deposits, such as a pattern of broken stripes or alternating “dashes.” The discontinuous deposits may also be, for example, island-like deposits such as circles, dots, etc., or deposits of irregular shapes. It should be appreciated that any desired pattern or shape of hook material deposits are within the scope and spirit of the invention.

The hook material 22 constitutes the male component of conventional hook-and-loop mechanical fastening systems and cooperates with a “loop” or loop-like material to define a releasable and re-attachable fastening system. Any number of commercially available and conventional micro-hook materials used in absorbent articles, including diaper attachment tabs, etc., may be used in the present invention. Conventional systems are, for example, available under the VELCRO trademark. The hook element may be provided by a single-prong hook configuration, a multiple-prong hook configuration or by a generally continuous, expanded-head configuration, such as provided by a mushroom-head type of hook element. The many arrangements and variations of such fastener systems are collectively known in the art as hook-and-loop fasteners.

Examples of hook-and-loop fastening systems and components are described in U.S. Pat. No. 5,019,073 issued May 28, 1991 to T. Roessler et al., the entire disclosure of which is hereby incorporated by reference in a manner that is consistent herewith. Other examples of hook-and-loop fastening systems are described in U.S. patent application Ser. No. 366,080 entitled HIGH-PEEL TAB FASTENER, filed Dec. 28, 1994 by G. Zehner et al. which corresponds to U.S. Pat. No. 5,605,735; and U.S. patent application Ser. No. 421,640 entitled MULTI-ATTACHMENT FASTENING SYSTEM, filed Apr. 13, 1995 by P. VanGompel et al.; the entire disclosures of which are hereby incorporated by reference in a manner that is consistent herewith. Examples of fastening tabs constructed with a carrier layer are described in U.S. patent application Ser. No. 08/603,477 of A. Long et al., entitled MECHANICAL FASTENING SYSTEM WITH GRIP TAB and filed Mar. 6, 1996 which corresponds to U.S. Pat. No. 5,624,429 which issued Apr. 29, 1997, the entire disclosure of which is hereby incorporated by reference in a manner which is consistent herewith.

In a particularly desirable embodiment of the invention, the hook material 22 is directly extruded onto the baffle material 14 to define laminated regions of the hook material 22 integral with the baffle material 14. Processes for directly extruding regions of a hook material directly onto a base material are described, for example, in U.S. Pat. Nos. 5,518,795; 5,260,015; and 5,744,080, all from Velcro Industries B.V. and incorporated herein in their entirety for all purposes. The extrusion process described in these patents may be used to directly deposit or embed the hook material 22 with the baffle material 14 in any desired pattern. The baffle material 14 is selected so as to be suitable for the extrusion process and to support the embedded hook material 22. From a manufacturing perspective, rolls of the baffle material 14 having the hook material already embedded therein may be supplied directly into an in-line processing line for producing the articles 10. The extruded embedded regions of hook material 22 also have a reduced thickness as compared to the hooked regions of conventional hook tape adhered to a base material.

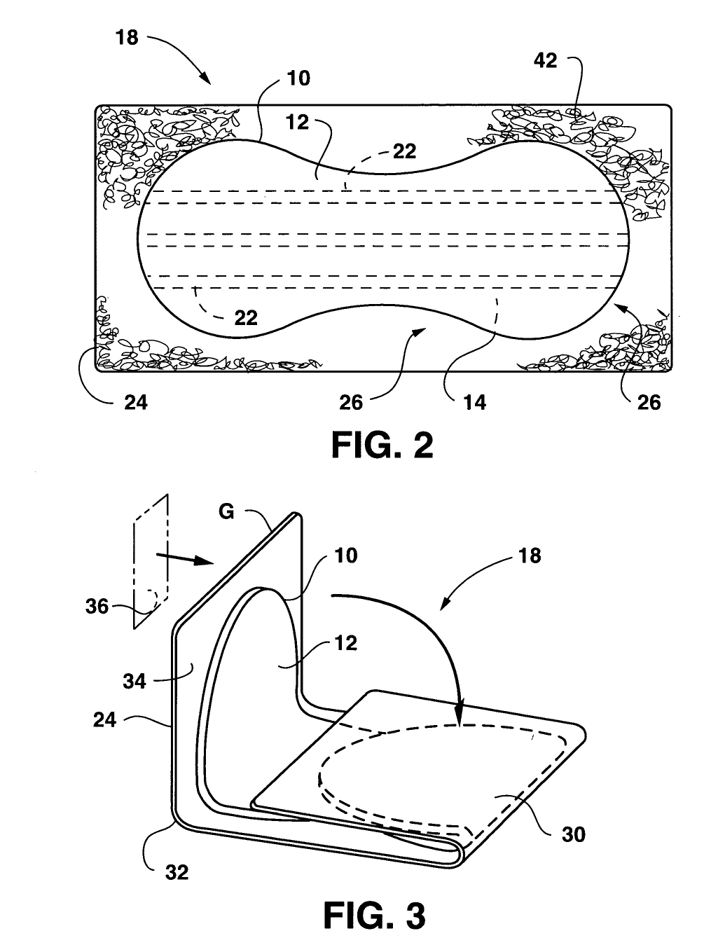

Referring to the figures in general, the backing sheet 24 is releasably attached to the garment facing side 16 of the baffle 14 and may have dimensions so as to extend beyond the lateral sides C, D and longitudinal ends A, B of the absorbent article 10 when attached to the baffle 14. For example, referring to the embodiment of FIG. 2, the backing sheet 24 may be generally rectangular with lateral sides E, F and longitudinal ends G, H that define a border 26 around the baffle 14. It should be appreciated that the backing sheet 24 may take on various shapes and sizes.

The backing sheet 24 may be formed wholly or in part by a hook compatible material, such as a nonwoven material 42, so as to cover and engage with the hook material 22 on the garment facing side 16 of the baffle 14. “Hook-compatible” material should be understood to encompass any material presenting a loop or loop-like surface for releasable attachment with the hook material 22. Suitable materials include, for example, a woven fabric, a nonwoven fabric, a knitted fabric, a perforated or apertured layer, and the like, as well as combinations thereof. Any number and combination of suitable cover materials may provide the “hook compatible” feature.

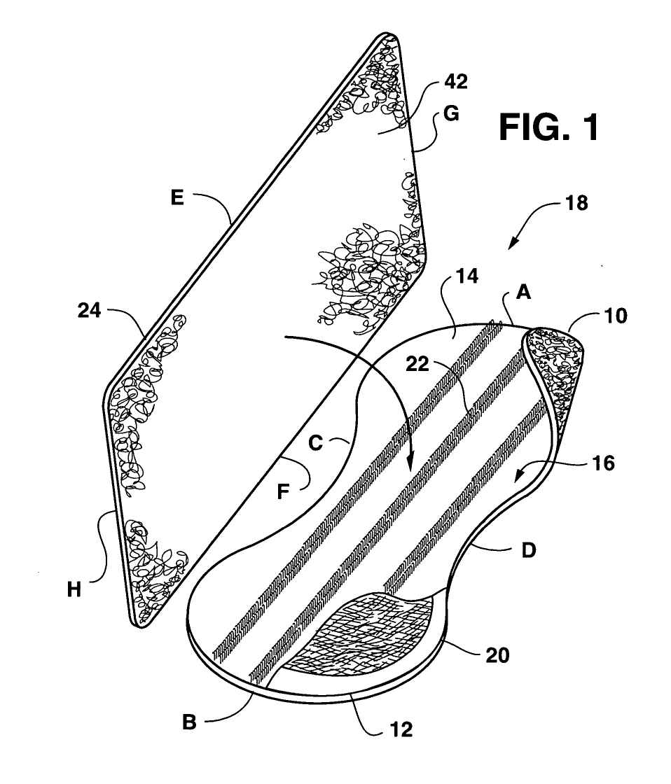

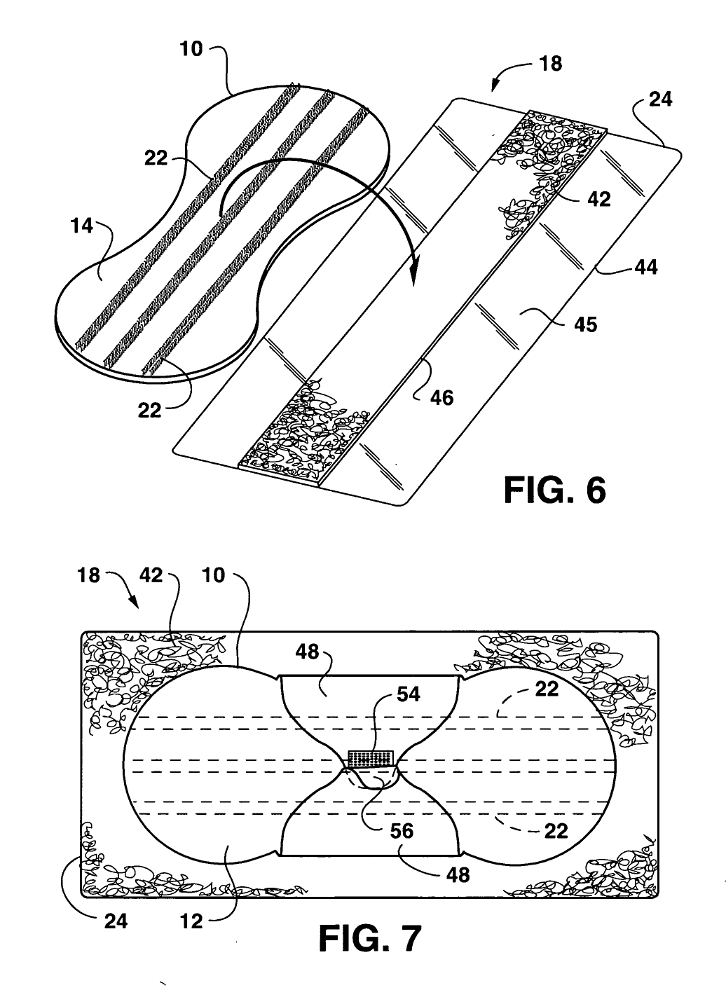

The backing sheet 24 may formed in its entirety of hook compatible material, such as the nonwoven backing sheet 24 of FIGS. 1 and 2. In alternate embodiments, the backing sheet 24 may be formed of other materials and include sufficient hook compatible material disposed for adequately attaching to the hook material 22 on the baffle 14. For example, in the embodiment of FIG. 6, the backing sheet 24 is formed of a base material 44, such as a film material 45, and a strip 46 of nonwoven material 42 is attached to the base material 44. The strip 46 has a width so as to extend across the pattern of striped hook material deposits 22 on the baffle 14. As discussed above, the use of a film base material 44 has particular advantages. For example, the film may be desirable for functionality and aesthetic purposes. The film may render the pouch liquid impermeable, and provide a suitable material for disposal of soiled articles.

In an alternate embodiment illustrated for example in FIG. 5, the backing sheet 24 is provided in the form of a laminated sheet of nonwoven material 42 and film material 45.

The film material 45 may include one or more layers of any conventional film material that is preferably liquid impermeable, but vapor permeable. As illustrated in the figures, the film material 45 defines the outward surfaces of the pouch 38 and may have any desired color or surface pattern. One type of film that may be used is a nonporous, continuous film that, because of its molecular structure, is capable of forming a vapor-pervious barrier. Among the various polymeric films which fall into this category include film made from poly(vinyl alcohol), polyvinyl acetate, ethylene vinyl alcohol, polyurethane, ethylene methyl acrylate, and ethylene methyl acrylic acid to make them breathable. If desired, it is also possible to add fillers to the film such as, for example, calcium carbonate and titanium dioxide, to increase opacity, decrease cost, and create a breathable film if the filled film is subsequently stretched. Another type of film which may be useful is a microporous film. These films have a number of interconnecting voids or holes which provide pathways for the transportation of water molecules from one surface to another.

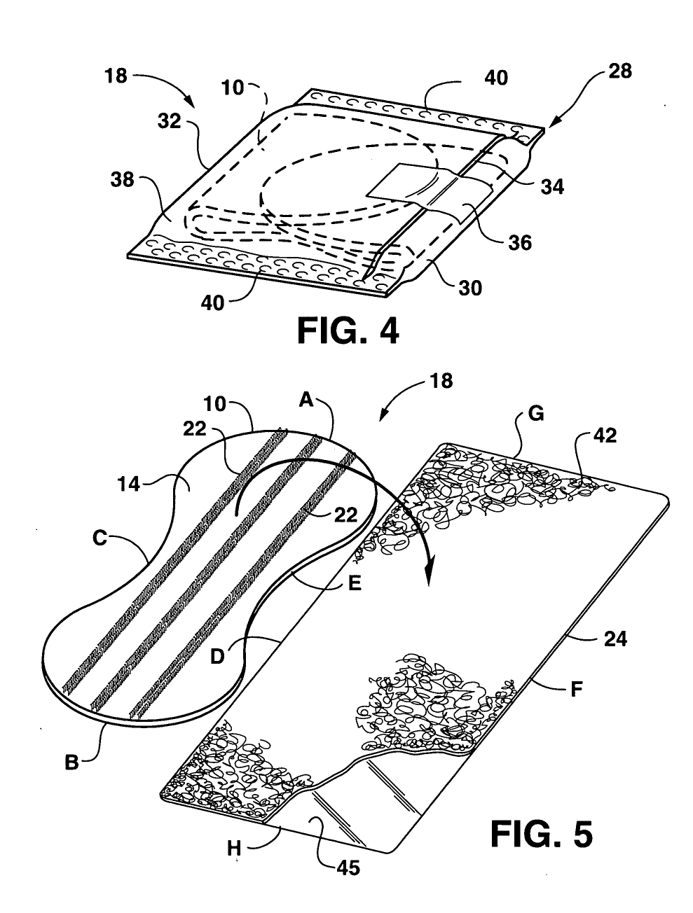

As can be seen from the figures, the backing sheet 24 is oriented such that the side thereof with the hook compatible material is disposed against the baffle 14 of the article 10. The article 10 and attached backing sheet 24 are then folded into a configuration such that the backing sheet 24 defines a pouch-like structure 38 for the absorbent article, as shown particularly in FIGS. 3 and 4. The size and shape of the pouch 38 will vary as a function of the size of the backing sheet 24, fold pattern, etc. In the illustrated tri-fold configuration, the backing sheet 24 and attached article 10 are folded at a first fold axis 30 and then at a second fold axis 32 to define the pouch 38 with flap 34. The adjacent lateral sides E, F of the backing sheet 24 define sides 40 of the pouch 38 that may be sealed or bonded together as illustrated in FIG. 4 by any conventional bonding or sealing technique. The seals can be made to be permanent seals, which means that the material adjacent to the seals will tear or break before the sealed layers separate. Alternately, the seals may be frangible seals, which means that the sealed layers can be separated or pulled apart.

In the illustrated tri-fold configuration, the longitudinal end G of the backing sheet 24 is folded over towards a front surface of the pouch 38 defined by the opposite longitudinal end portion H to define the opening flap 34. This flap 34 may be sealed or otherwise releasably attached to the front surface of the pouch 38, for example by way of an adhesive tab 36 as illustrated in FIG. 4.

By releasably attaching to the baffle hook material 22, the backing sheet 24 protects the top cover 12 from being engaged by the hook material 22 when the article is folded or unfolded by the wearer prior to use. Engagement of the hook material 22 with the cover 12 is undesirable in that it may result in damage (i.e., tears or holes) or picking of the cover material and give an overall impression of lesser quality to the wearer. The backing sheet 24 additionally provides a means for defining a convenient and discrete individual pouch 38 for the article 10. For use, the wearer simply opens the flap 34, unfolds the pouch 38, peels the pouch material (backing sheet 24) from the baffle side of the article 10, and presses the article 10 into place in the crotch region of the undergarment.

The invention is also useful with absorbent articles 10 that incorporate laterally extending wings 48 configured to fold around the crotch portion of a wearer's undergarment. Such articles 10 are illustrated in FIGS. 7, 8, and 9. The wings 48 may be folded onto the top cover 12 prior to attaching the article 10 to the backing sheet 24 such that the sides of the wings 48 that face the undergarment in use of the article 10 are outwardly facing. As with the embodiment of FIG. 9, the wings 48 may have hook material 58 provided in a pattern on the garment facing side thereof for attachment to the undergarment, similar to the hook material 22 provided on the baffle 14. Alternately, the wings may overlap when folded onto the top cover 12 and the wing hook material 58 may be used to attach overlapping portions of the wings 48 together in the folded configuration on top of the cover and after being folded around the undergarment. In this case, a protective material sheet 50 may be attached to cover the wing hook material 58, as illustrated in FIG. 9, prior to folding the article 10 and backing sheet 24 so that the wing hook material 58 does not engage with the top cover 12.

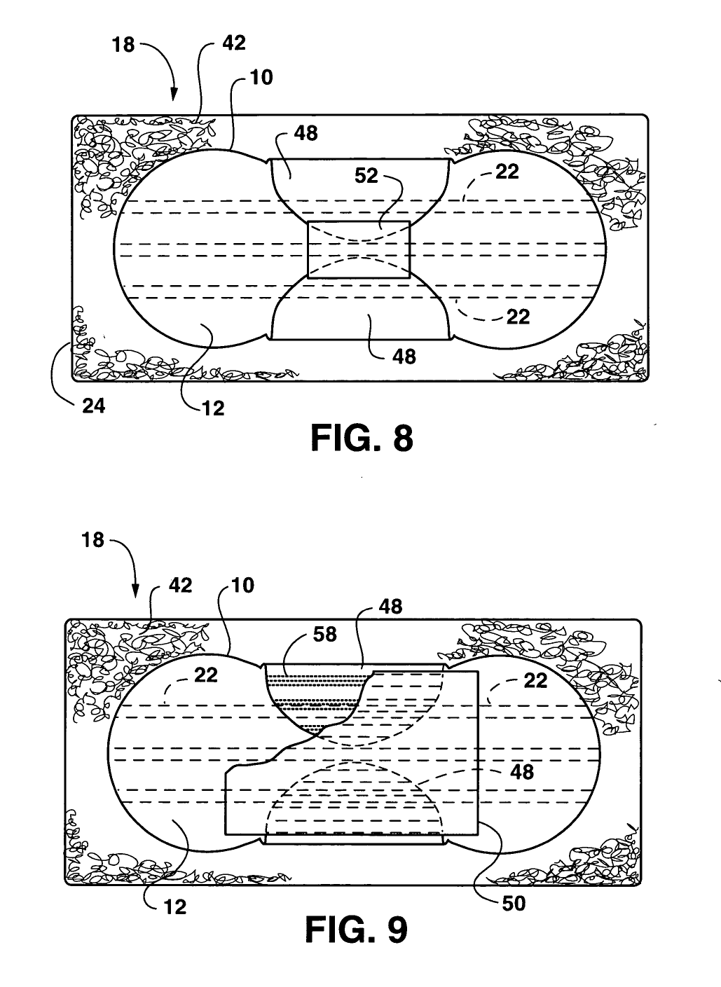

In the embodiment of FIG. 8, the wings 48 are folded onto the top cover 12 and secured together with an adhesive tap 52. Although not illustrated, the wings 48 may include adhesive regions on the garment facing side for attaching the wings 48 to the wearer's undergarment and/or to each other. A peel strip may be provided on the adhesive regions.

In the embodiment of FIG. 7, the wings have overlapping portions wherein a deposit of adhesive or hook material 54 releasably attaches to an overlapping tab 56. This attachment mechanism may also be used to attach the wings 48 in place around the wearer's undergarment.

As mentioned, with the combination pouch 38 also provides a means to discretely dispose of soiled articles 10 without the need for disposal wrappers, pouches, or the like, in additional to a peel strip. After unfolding the pouch 38 and peeling the baking sheet 24 from the article, the wearer can simply grasp the soiled article by the edges, press it against the backing sheet 24 and fold the combination into a disposal pouch. A film sided backing sheet may be desirable in that it may reduce odor from the soiled product. Any suitable sealing device, such as a peel strip, adhesive patch, etc., may be provided to hold the pouch (with soiled article inside) closed. This sealing device may be, for example, the flap attachment device 36 used to keep the flap 34 attached to the pouch (FIGS. 3 and 4).

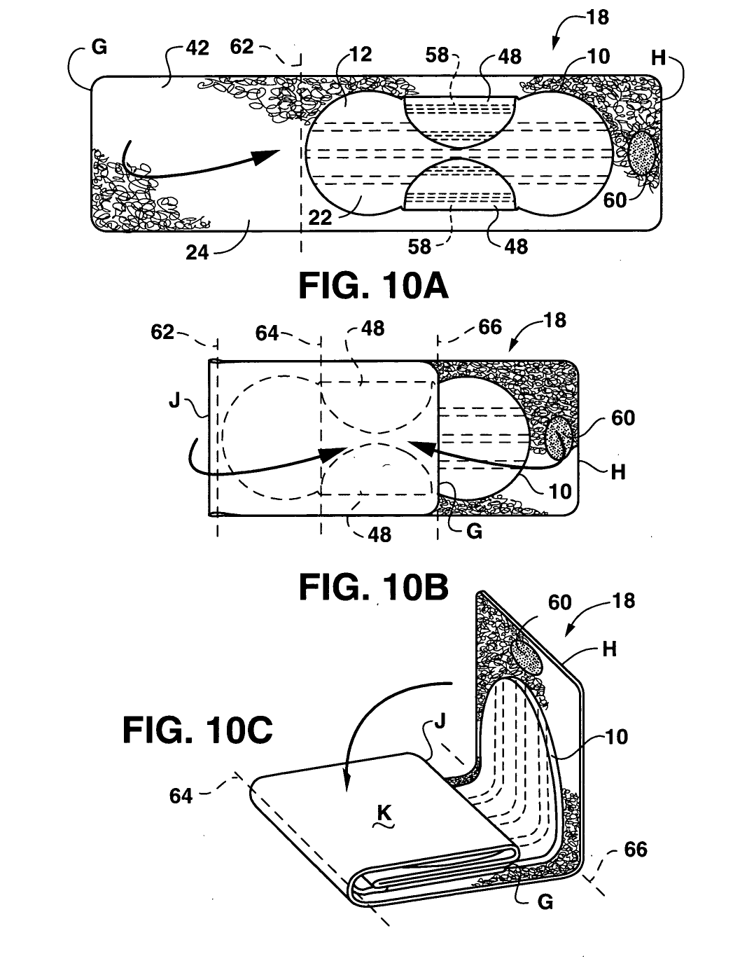

FIGS. 10A through 10C illustrate an alternate embodiment wherein the backing sheet 24 has an extended longitudinal length as compared to the absorbent article 10. This embodiment may be particularly useful for articles 10 having wings 48 with hook material 58 provided thereon. The wings 48 are folded onto the top cover 12 of the article 10, and may be overlapping. The article 10 is placed generally at one longitudinal end of the backing sheet 24, and the sheet 24 is folded at fold line 62 such that the extended portion of the sheet 24 folds back over the article 10 and covers the wings 48, as depicted in FIG. 10B. In this regard, the wings 48 (with hook material 58) are covered as in the embodiment of FIG. 9, but without a separate protective material. The longitudinal end G of the backing sheet extends at least past the wings 48, and may extend to the opposite longitudinal end H. The combination may then be folded into a pouch, as depicted in FIG. 10C. The package is first folded at fold line 64 such that the intermediate end J is brought to overlie the end G of the backing sheet 24. The opposite end of the combination is then folded over at fold line 66 to complete the pouch. After this, the sides of the pouch may be sealed as described above. A hook material patch 60 may be provided at the longitudinal end H of the backing sheet 24 to secure the folded over flap of backing sheet 24 against the outer surface K of the pouch. The patch 60 may also serve to seal the pouch when wrapping and disposing a soiled article within the backing sheet 24, as described above.

It should be apparent to those skilled in the art that various modifications and variations can be made to the embodiments of the invention described and illustrated herein without departing from the scope and spirit of the claims as set forth in the appended claims and their equivalents.

1. A packaged absorbent article for placement in the crotch region of an undergarment, comprising:
a generally liquid permeable top cover;
a generally liquid impermeable baffle having a garment facing side;
an absorbent structure disposed between said top cover and said baffle;
said garment facing side of said baffle comprising hook material defined in a pattern thereon;
a hook compatible backing sheet releasably attached to said baffle, said backing sheet comprising dimensions so as to extend beyond lateral sides and longitudinal ends of said absorbent article when attached to said baffle;
said article and attached backing sheet folded into a configuration such that lateral sides of said backing sheet are brought together and said backing sheet defines a pouch for said article.

2. The packaged absorbent article as in claim 1, wherein said hook material on said garment facing side of said baffle provides a primary attachment mechanism between said article and the undergarment.

3. The packaged absorbent article as in claim 1, wherein said top cover comprises a hook compatible material, said backing sheet preventing attachment of said hook material to said top cover in said folded configuration of said article.

4. The packaged absorbent article as in claim 1, wherein said article and attached backing sheet are folded in a tri-fold configuration.

5. The packaged absorbent article as in claim 4, wherein one longitudinal end of said tri-fold configuration defines an opening flap disposed against a front outwardly facing surface of said pouch.

6. The packaged absorbent article as in claim 5, wherein said opening flap is releasably attached to said outwardly facing side of said pouch.

7. The packaged absorbent article as in claim 1, wherein said lateral sides of said backing sheet are attached together to define sealed sides of said pouch.

8. The packaged absorbent article as in claim 7, wherein one longitudinal end of said backing sheet is folded over onto a front surface of said pouch and defines an opening flap.

9. The packaged absorbent article as in claim 1, wherein said backing sheet comprises a nonwoven material that is releasably attachable to said hook material.

10. The packaged absorbent article as in claim 1, wherein said backing sheet comprises a first side of film material and an opposite side of a hook compatible material, said film defining outwardly facing surfaces of said pouch.

11. The packaged absorbent article as in claim 9, wherein said backing sheet comprises a laminate of said film material and a nonwoven material.

12. The packaged absorbent article as in claim 10, wherein said hook compatible material is bordered at least partially by said film material.

13. The packaged absorbent article as in claim 12, wherein said hook compatible material comprises a longitudinally oriented strip attached to said film material.

14. The packaged absorbent article as in claim 1, wherein said article further comprises opposite laterally extending wings configured to fold around the crotch portion of a wearer's undergarment, said wings folded onto said top cover in said folded configuration of said article such that garment facing sides of said wings are outwardly facing.

15. The packaged absorbent article as in claim 14, wherein said wings comprise hook material provided on said garment facing sides for releasable attachment of said wings to the wearer's undergarment, and further comprising a protective material sheet releasably attached to said wing hook material in said folded configuration of said article.

16. The packaged absorbent article as in claim 14, wherein said wings at least partially overlap on said top cover and are releasably attached to each other.

17. The packaged absorbent article as in claim 16, wherein at least one of said wings comprises a hook material that releasably engages with said other wing in said folded configuration of said article.

18. The packaged absorbent article as in claim 16, wherein said wings are releasably attached by an adhesive.

19. The packaged absorbent article as in claim 14, wherein said wings comprise hook material provided on said garment facing sides for releasable attachment of said wings to the wearer's undergarment, said backing sheet comprising a longitudinally extending portion that is folded over said wings prior to folding said article and backing sheet into said pouch.

20. The packaged absorbent article as in claim 20, wherein said article and backing sheet are tri-folded into said pouch.

22. A packaged absorbent article for placement in the crotch region of an undergarment, comprising:
a generally liquid permeable top cover;
a generally liquid impermeable baffle having a garment facing side;
an absorbent structure disposed between said top cover and said baffle;
said garment facing side of said baffle comprising hook material defined in a pattern sufficient for said hook material to provide a primary attachment mechanism between said article and the undergarment;
a backing sheet comprising a first side of nonwoven material releasably attached to said baffle hook material, and an opposite side having different hook engagement properties as compared to said first side; and
said article and attached backing sheet folded into a pouch configuration such that said film material is outwardly facing.

23. The packaged absorbent article as in claim 22, wherein said opposite side comprises a film material.

24. The packaged absorbent article as in claim 22, wherein said opposite side comprises a nonwoven material.

25. The packaged absorbent article as in claim 22, wherein said backing sheet extends laterally beyond said baffle and is bonded to itself along lateral sides of said pouch configuration.

26. The packaged absorbent article as in claim 22, wherein said backing sheet comprises a laminate of said nonwoven material and a different material.

27. The packaged absorbent article as in claim 22, wherein said article and attached backing sheet are folded into said pouch configuration such that one longitudinal end of said backing sheet defines an opening flap disposed against a front outwardly facing surface of said pouch.

28. The packaged absorbent article as in claim 27, wherein said opening flap is releasably attached to said outwardly facing side of said pouch.
